(19) United States
(12) Patent Application Publication
(10) Pub. No.: US 2020/0303075 A1
(43) Pub. Date: Sep. 24, 2020

(54) SYSTEM AND A METHOD TO PREDICT OCCURRENCE OF A CHRONIC DISEASES

(71) Applicants: Kundan Krishna, Fremont, CA (US); Naveen Koul, San Jose, CA (US); Dilip Kumar Maurya, Pondicherry (IN); Shivata Chowdhry, San Jose, CA (US)

(72) Inventors: Kundan Krishna, Fremont, CA (US); Naveen Koul, San Jose, CA (US); Dilip Kumar Maurya, Pondicherry (IN); Shivata Chowdhry, San Jose, CA (US)

(21) Appl. No.: 16/356,168

(22) Filed: Mar. 18, 2019

(51) Int. Cl. G16H 50/30; G16H 50/20; G16H 50/70

(52) U.S. Cl. CPC G16H 50/30; G16H 10/40; G16H 50/70; G16H 50/20

(57) ABSTRACT

The present invention provides a method for predicting occurrence of a chronic diseases in a patient at an early stage using a trained Deep Neural Network (DNN) using the patient's routine or preventive pathological test result data. The invention collects the past routine/preventive laboratory test results diagnosed with the chronic disease and trained a DNN using the labelled data. The embodiment of present invention that warning or predicting of a chronic disease in a patient comprising the steps of Collecting the patient's historical routine/preventive pathological test result data who are suffering from a chronic diseases; Pre-processing of collected data; training a Deep Neural Network (DNN) with the preprocessed data and feed a new patient similar set of routine/preventive pathological test result data to provide an estimate of the early detection of a chronic diseases.

### FIELD OF THE INVENTION

The present invention relates to a method for using an artificial neural network for medical prognosis, and more particularly, to a method for using pathological test result data to create a prediction score for predicting occurrence of a chronic disease.

### BACKGROUND

Chronic diseases are long-lasting conditions that are mostly non-curable but can usually be controlled. People with chronic disease have to manage their day to day activity for regulating symptoms that can affect their quality of life, or shorten their life expectancy. According to various statistical data, chronic diseases are the major causes of premature death around the world. People with chronic disease often think that they are free from the disease when they have no symptoms. Having no symptoms, however, does not necessarily mean that chronic disease has disappeared.

Though chronic disease are incurable, but through effective behavior change efforts and appropriate medical management, chronic diseases and their consequences can often be prevented or managed effectively. If a chronic disease gets detected at earlier stage, a more effective treatment regime is possible. However, even with current advance diagnosing techniques, detecting a chronic disease at earlier stage is not possible. There are many chronic diseases such as Liver Cirrhosis, asthma, different types of Cancer, diabetes, ischemic Heart diseases and strokes etc. which are almost impossible to detect in its early stage. This late detection minimizes the chance of survival for a patient even at the cost of huge financial burden. Early warning and diagnosis of chronic diseases is essential to maximize the possibility of successfully treating the disease. Medical professionals are left with fewer options to provide treatment and mostly fail because the diseases have reached to its peak or incurable stage in the last phase.

Current standard diagnostic techniques are not effective for early detection of chronic disease. In some healthcare providers, algorithms with certain rules are utilized to categorize patients into high risk and low risk group. They compare the biomarker values of a patient with reference biomarker values and when the value reaches above a threshold point, an alert is generated and the patient is categorized into a high-risk category. In other conventional methods, the healthcare providers use a combination of different biomarkers that are associated with the onset of a chronic disease to diagnose a patient. These techniques are not effective in predicting occurrence of a chronic disease at early stages. Moreover, the symptoms associated with onset of a chronic disease are very gradual and often they go undetected till late stages of the disease. Hence, the diseases have reached to its peak or incurable stage in the last phase and there have been many such cases where patients did not survive despite of the huge financial investments.

There is therefore, a need for an inventive approach that can early predict or provide a warning signal to medical professionals and patient about the possibility of a chronic disease which does not exist at present but may occur in future. The present invention describes a method to early warn or predict any potential chronic diseases a patient might be at risk of developing.

### SUMMARY

The present invention provides a method for detecting any chronic diseases in an early stage with the input of patient routine or preventive pathological test result data. Most of the time, patient goes through routine pathological laboratory tests for blood/serum analysis, urine analysis and stool analysis under preventive health check-up programs. Some of these laboratory measures changes over a period until patient are confirmed with a chronic disease. Such patient's historical routine laboratory test results data is then used to train a Deep Neural Network (DNN) to analyze and identify a correlation between the pattern of change in different components of pathological test result over a period of time and occurrence of a chronic disease at later stage. The correlation identified by the Deep Neural Network is then used to generate a prediction score for a user when his historical pathological test result data is feed into the Deep Neural Network. The Deep Neural Network analyze the pattern of change in historical pathological test result data of the user and compare with the correlation learned by the Deep Neural Network to generate a prediction score that predicts the occurrence of the chronic disease.

In an aspect of a present invention, a system for predicting occurrence of a chronic disease in a patient using historical pathological test result data is provided. The system comprising: a database for storing a dataset of historical pathological test result data of a plurality of patients diagnosed with a chronic disease; a server having a Deep Neural Network in communication with the database, the DNN extracts the dataset from the database to identify a correlation between the changes in historical pathological data of a patient and the chronic disease; a user interface to feed a new patient historical test result data into the trained Deep Neural Network; wherein trained Deep Neural Network analyze change in historical test result data of the new patient data with the correlation to identify a prediction score for probability of occurrence of the chronic disease; an application interface in the server to notify a user the prediction score. The training of Deep Neural Network is through supervised learning and the dataset to train the Deep Neural Network are Labeled data. The dataset for training is pre-processed before inputting into the Deep Neural Network, wherein the pre-processed steps comprises Normalization and scaling of the dataset. The changes in historical pathological data includes changes in one or more analytes or parameters in blood/serum of the patient, said one or more analytes or parameters in blood/serum comprises blood components, CBC, KFT, LFT, lipid profile parameters.

In another aspect of present invention, a method for predicting occurrence of a chronic disease in a patient using historical pathological test result data is provided. The method comprises: training a Deep Neural Network with a dataset comprising historical pathological test result data of a plurality of patients that have been diagnosed with a chronic disease, said Deep Neural Network analyze the data and learns to identify a correlation between the changes in historical pathological data of a patient and the chronic disease; feeding a new patient historical test result data into the trained Deep Neural Network wherein trained Deep Neural Network analyze change in historical test result data of the new patient data with the correlation to identify the probability of occurrence of the chronic disease. The training of Deep Neural Network is through supervised learning and the dataset to train the Deep Neural Network are Labeled data. The dataset for training is pre-processed before inputting into the Deep Neural Network, wherein the pre-processed steps comprises Normalization and scaling of the dataset. The changes in historical pathological data includes changes in one or more analytes or parameters in blood/serum of the patient, said one or more analytes or parameters in blood/serum comprises blood components, CBC, KFT, LFT, lipid profile parameters.

### BRIEF DESCRIPTION OF DRAWINGS

The preferred embodiment of the invention will hereinafter be described in conjunction with the appended drawings provided to illustrate and not to limit the scope of the invention, wherein like designation denotes like element and in which:

FIG. 1 is flow diagram illustrating a method to detect the probability of occurrence of a chronic disease based on a user's historical preventive or routine pathological test result data, in accordance with an embodiment of present invention.

FIG. 2 illustrates a learning method for training Deep Neural Network for detecting a chronic disease, in accordance with an embodiment of the present invention.

FIG. 3 is a process flow illustrating a system for using a person preventive or routine diagnostic report data for predicting a probability of occurrence of a chronic disease, in accordance with an embodiment of the present invention.

### DETAILED DESCRIPTION

In the following detailed description of embodiments of the invention, numerous specific details are set forth in order to provide a thorough understanding of the embodiments of the invention. However, it will be obvious to a person skilled in art that the embodiments of the invention may be practiced with or without these specific details. In other instances, well known methods, procedures and components have not been described in details so as not to unnecessarily obscure aspects of the embodiments of the invention.

Furthermore, it will be clear that the invention is not limited to these embodiments only. Numerous modifications, changes, variations, substitutions and equivalents will be apparent to those skilled in the art, without parting from the spirit and scope of the invention.

The present invention works in the field of medical prognosis where the patients routine or preventive pathological tests result data are utilized to predict occurrence of a chronic disease in the patient. The present invention provides a method for warning or predicting of chronic diseases in an early stage with the input of patient routine or preventive pathological test result data. The warning or predicting of a chronic disease in a patient comprising the steps of (1) Collecting the patient's historical routine/preventive pathological test result data who are suffering from a chronic diseases (2) Pre-processing of collected data (3) training a Deep Neural Network (DNN) with the preprocessed data (4) feed a new patient similar set of routine/preventive pathological test result data to provide an estimate of the early detection of a chronic diseases. Any of the steps mentioned above can be deployed as a single module or combined with any other module(s) in an embodiment.

The invention utilize historical or preventive pathological test result data of a patient diagnosed with a chronic disease as an input to a Deep Neural Network which can then later be used to predict the occurrence of a chronic disease for any new patient using their pathological test results data. The prediction of occurrence of a chronic disease may include but is not limited to prediction of neurological disorder, lungs disease, liver related disorder, cancer, gastrointestinal disorder, blood based disorder, heart related disorder etc.

The present invention provides a method for detecting the possibility of a chronic disease in an early stage with the input of patient routine or preventive pathological test result data. The routine preventive pathological test result include test result data on blood/serum analysis, urine analysis and stool analysis. The blood analysis result data may comprise test related to chemical pathology, hematology, anatomical pathology, medical microbiology, immunopathology, genetic pathology, genetic pathology, general pathology or clinical pathology. The blood analysis parameters may comprise but are not limited to CBC, CMP, LIPID Panel, TSH quantification, LFT, iron studies etc.

The urine analysis test result data comprise but are not limited to ketoacids, electrolytes, acids, alkalines, proteins, glucose or amount of pathogen. The stool test pathological test result data may comprise but are not limited to microscopic examination, chemical tests and microbiological test.

The patient undergoes pathological laboratory tests time to time as a part of preventive measure. Some of those patients later start suffering from a chronic disease. The system collects those patients past routine/preventive laboratory test results of that have been diagnosed with chronic disease and the data is Labeled with their current suffering chronic diseases. Collected data also carries some of patient information includes but not limited to age, gender and weight etc. Such patient's historical routine laboratory test results data can be learned by a Deep Neural Network to identify correlation between change pattern in pathological test result parameter and the onset of a chronic disease.

The patient historical routine laboratory test result data may include a variety of pathological test data which have some association with likelihood of occurrence of a chronic disease. Such data may be derived from measurement of any blood analytes or parameters that include, but are not limited to, endocrine substances such as hormones, exocrine substances such as enzymes, and neurotransmitters, electrolytes, proteins, carbohydrates, growth factors, cytokines, fatty acids, triglycerides, and cholesterol.

The present invention provides a significant advantage to a patient by disclosing a method that can warn the patient at early stage of chronic diseases so that patients can start treatments as early as possible to maximize their outcomes against the deadly diseases. With the development of the method disclosed here, screening and diagnosis of chronic diseases will become relatively non-invasive.

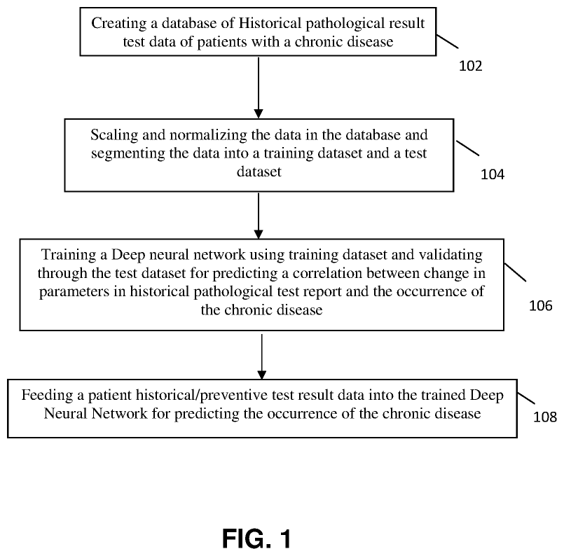

FIG. 1 is flow diagram illustrating a method to detect the probability of occurrence of a chronic disease based on a user's historical preventive or routine pathological test result data, in accordance with an embodiment of present invention. The method firstly involves creating a database of routine pathological test result data of patients with a chronic disease, step 102. The data of patients with a chronic disease is collected from different diagnosis center, hospitals, medical institutions etc. and aggregated to form a central database. The database contains blood or serum test result for various parameters or compounds in the sample of patients who have diagnosed with the chronic disease. The routine preventive pathological test result data may contain results on blood/serum test, urine analysis and stool analysis, and include tests related to chemical pathology, heamatology, anatomical pathology, medical microbiology, immunopathology, genetic pathology, genetic pathology, general pathology or clinical pathology. The patient historical pathological test result data provides an insight to change in the blood/serum parameters over a range of time till the chronic disease is detected. During the patient life, the patient undergoes preventive or routine pathological tests to ascertain a healthy life. Routine tests at different stages show different parameters values, until the chronic disease is detected, which means that occurrence of a chronic disease followed a specific pattern in change of blood serum parameters. The database can be accessed by a central server through internet or local area network. The central database electronically stores chronic disease information and enables a user to access the stored information. After the database is created, at step 104, the data having different parameters and values are scaled and normalized in the range of 0 to 1. The normalized data is then segmented into a training dataset and a test dataset. At step 106, the training dataset is fed into a Deep Neural Network, so as to train the Deep Neural Network to learn and find a correlation between the changes in different parameters values over a period of time till the occurrence of the chronic disease. The trained Deep Neural Network (DNN) is then tested using the test dataset for validating the output results. The training of Deep Neural Network is based on supervised learning, wherein the input data and output values are Labeled and the DNN is trained to identify the correlation between them. The training process of the Deep Neural Network is continued until its training error comes under less than 1%. The trained DNN is verified with the test datasets to check its prediction success rate. If prediction success rate reaches to an accuracy of around 99%, then the DNN model is saved for later prediction task over a new patient data. If the error is high, then training and verification continues.

In an embodiment, the historical pathological datasets of the patients who have been confirmed to be suffered from a chronic disease is Labeled with the corresponding chronic disease. A DNN training method such as stochastic gradient method is used for mapping input historical pathological datasets to the Labeled output. While doing so, a DNN architecture is trained with its learned parameters which ultimately act as a trained classifier to provide a prediction score on a new users pathological test result data to represent the probability of suffering from that particular chronic disease. During the training stage, the DNN process the Labeled input data with the output data to identify a correlation between change in patterns in different components of pathological test result data and the onset of chronic disease. The identified correlation index is then used on a new patient test data to predict the occurrence of the chronic disease.

The trained DNN at is then used to predict the occurrence of the chronic disease on new patient data at step 108. The patients historical preventive or routine test data is input in the trained DNN and based on the training algorithm, the DNN predicts the occurrence of the chronic disease to the patient. The trained DNN analyze the pattern in patients historical pathological test result data with the correlation index learned during the training stage, and DNN generate a prediction score that signifies the probability of the patient to have the chronic disease in near future.

In an embodiment of present invention, the trained DNN network either resides in a server or is distributed as software installed on the client devices. The pathological test result data of the patient to be examined for the probability of having a chronic disease is entered through a user interface present on the client device. In an embodiment of present invention, the historical pathological test result data of the user can be entered using a user interface or it can be shared by the diagnostic centers. The user interface displays the end result of the analysis to the user in form of the prediction score.

In another embodiment, the prediction score of the patient showing the probability of occurrence of the chronic disease is notified to the patient, physicians or health care providers through notification means, such as email, sms etc. A mobile application can be installed on a user's mobile device which is connected to the server that stores the trained DNN model and the mobile application can serve as a user interface to interact with the trained DNN model.

In an embodiment of present invention, the system and method notifies the physician, the healthcare provider and the patient about the probability of occurrence of the chronic disease. This will help the physician to formulate an effective treatment regime in good time for the treatment of high risk patient. In an embodiment, the system can notify the patient, the physician and the health care provider by sms or through a mobile application connected to the internet. This provide a support system for both patient and physician by improving the monitoring of a patient's health status and preventing further deterioration of the patient through chronic disease management. It also enables a patient to design individualized treatment action plan for the chronic disease.

### DNN Architecture

Deep Neural Networks are stacked neural network composed of several layers of neural networks. Neural networks are a set of algorithms, modeled loosely after the human brain, that are designed to recognize patterns. They interpret sensory data through a kind of machine perception, labeling or clustering raw input. Different layers may perform different kinds of transformations on their inputs. Signals travel from the first layer (the input layer), to the last layer (the output layer), possibly after traversing the layers multiple times. In deep-learning networks, each layer of nodes trains on a distinct set of features based on the previous layer's output. As the signal advances from previous layer to next layer, the more complex the features, the next node can recognize, since it aggregate and recombine features from the previous layer.

The Deep Neural Network (DNN) is an artificial neural network (ANN) comprising a plurality of artificial neuron with multiple layers between the input and output layers. The DNN finds the correct mathematical manipulation to turn the input into the output, whether it is a linear relationship or a non-linear relationship. Deep learning is an aspect of artificial intelligence (AI) that is concerned with emulating the learning approach that human beings use to gain certain types of knowledge. At its simplest, deep learning can be thought of as a way to automate predictive analytics. While traditional machine learning algorithms are linear, deep learning algorithms are stacked in a hierarchy of increasing complexity and abstraction.

### Creating a Database of Historical Routine/Preventive Pathological Test Result Data

Individuals undergo routine or preventive laboratory tests during various stages in life. These tests are being performed at various diagnostics centers where serum/blood samples, urine samples and stool samples of individuals are taken and investigated in their laboratories. The pathological test determines the cause and nature of diseases by examining and testing body fluids (blood and urine samples) and stool samples. An investigation report is then delivered to the patient detailing the presence of different analytes in the serum or blood. A healthcare record for each patient is then saved in the diagnostic center database. The health care record may include medical history, notes and other information about health including the symptoms, diagnoses, medications, lab results, vital signs, immunization and reports from pathological tests. Health records aim to store a patient's health information in one place, providing quick access when required.

The health records of different individuals are aggregated to create a master database with health records of multiple individuals. The aggregation of health records can be carried out in number of ways. In one way, a central server on a request extracts the health records from different diagnostic centers, medical centers and institutions. The database can also be created by manually putting the health records of different patients in the database. A number of individual components are contained within the collective patient database; reflecting the diverse nature of a patient's healthcare record. While each individual medical discipline and its associated data would be represented in the generalized strategy, embodiments of invention segments the health record information of each patients into type of pathological test result data, age, demographic information, gender, Medical history and disease/problem list, current presentation and complaints etc.

In the database, pathological test result database of those patient who have developed a chronic disease in later stages are identified and a specific sub-database of patients with a chronic disease is created for each chronic disease. Since, there are different chronic diseases, therefore, sub-database of patients with each chronic disease is created separately. Each of the specific sub-databases contains health records of those patients who are suffering from that chronic disease. Collected data also carries some of patient de- identified information but not limited to age and weight. The database electronically stores chronic disease information and patient historical pathological test result data and enables a user to access the stored information.

### Pre-Processing of Collected Data

The database of historical/preventive routine pathological data has various parameters and is of patient with different number of diseases or diseases-free individuals. The data in the database is pre-processed before feeding into the Deep Neural Network. The step of pre- processing of collected data involve: data segregation, data partitioning, selection of data size and data de-duplication. The data segregation steps involve segregating the data of different individuals as per the chronic disease associated with patients. This implies that data of patients with a particular chronic disease is stored in a separate sub-dataset. Each type of chronic disease has a separate sub-dataset of those patients' historical pathological result test data that have been diagnosed with corresponding chronic disease. During data partitioning, the data is split into training set and test set. The training dataset is used to train the Deep Neural Network (DNN). It is the set in which the algorithm is run. The test set is then run on the trained DNN; the test set provides an unbiased performance evaluation of the trained DNN model on unseen data. Trained DNN is verified with test datasets to check the prediction success rate. If the prediction success rate reaches to an accuracy of around 99%, then the trained model is saved for later prediction task over a new patient data. If the error is high, then training and verification continues.

The data size for training dataset has to be large in the size, so that the DNN can be trained effectively to derive the relationship between different parameters of the pathological test and the probability of occurrence of a chronic disease. The test dataset should be large enough to indicate the overall performance of final trained DNN model. The data collected in the database of historical pathological reports of the individuals is processed to remove any duplicate data. The removal of duplicate data ensures the accurate performance of the DNN model. The duplicate data is removed to eliminate any probability of two similar data getting segregated into training data set as well as test data set, thus removing any possibility of false positives.

The dataset in sub-database are also processed to transform the raw data into a format such that deep learning can best learn from the data. These pre-processing techniques involve: scaling the data into matrixes, normalizing the parameters independently to have a mean 0 and a standard deviation of 1. The other techniques that can be used for converting raw data into format suitable for DNN can be: Embedding wherein the text data is represented with dense vectors learned in unsupervised way; Imputation wherein the missing values in data are denoted with a character; Label encoding etc.

### Training a Deep Neural Network (DNN) with the Preprocessed Data

The pre-processed training dataset for patients with different chronic disease are then feed into the Deep Neural Network for training purpose. The patient historical pathological dataset who have been confirmed to be suffering from a chronic disease is Labeled with the corresponding chronic disease. DNN training method such as stochastic gradient method is then used for mapping input historical pathological datasets to the Labeled output. While doing so a DNN architecture is found with its learned parameters which ultimately act as a trained classifier to analyze a correlation between the pattern changes in components of the pathological test results and the onset of the chronic disease. The method utilized supervised learning method for training DNN with the help of pathological training datasets. The parameters of the pathological test result and the chronic disease are Labeled in the training data. The training data consist of a set of training examples. The learning is based on available training data which are essentially sets of input values paired with the known output results. In this training method, the learning examples are a pair of an input vector and a desired output vector. The learning algorithm analyzes the training data and produces and inferred function which can then be used for mapping later data.

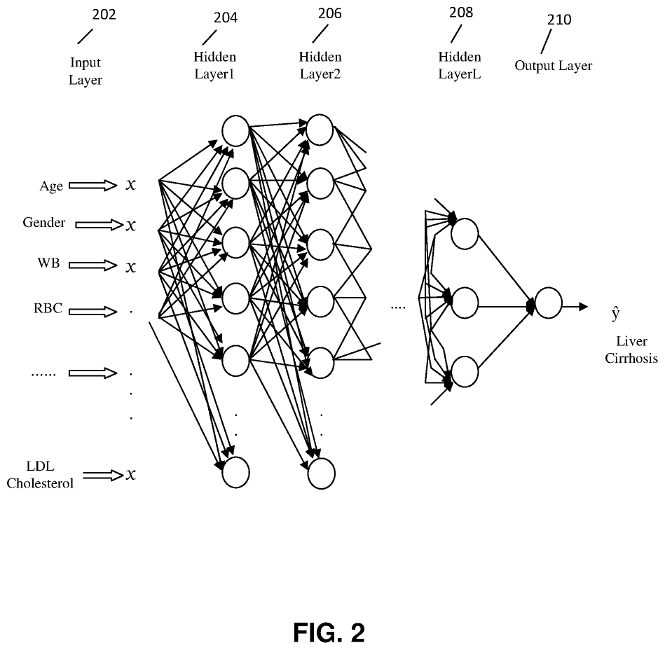

FIG. 2 illustrates a learning method for training Deep Neural Network for detecting a chronic disease, in accordance with an embodiment of the present invention. In the figure, Deep Neural Network is trained for predicting the abnormalities associated with Liver Cirrhosis disease. The Deep Neural Network comprise an Input layer 202, an output layer 210 and a plurality of Hidden layers (Hidden Layer 1 (204), Hidden layer 2 (206)—Hidden Layer L (208)). The pre-processed dataset containing historical pathological test result data of those patients who have been diagnosed with the chronic disease associated with Liver Cirrhosis is feed into the Input layer 202 of Deep Neural Network. In an embodiment, the routine preventive pathological test result includes test result data on blood/serum analysis, urine analysis and stool analysis. The blood analysis result data may comprise test related to chemical pathology, hematology, anatomical pathology, medical microbiology, immunopathology, genetic pathology, genetic pathology, general pathology or clinical pathology. The blood analysis parameters may comprise but are not limited to CBC, CMP, LIPID Panel, TSH quantification, LFT, iron studies etc.

The urine analysis test result data comprise but are not limited to ketoacids, electrolytes, acids, alkalines, proteins, glucose or amount of pathogen. The stool test pathological test result data may comprise but are not limited to microscopic examination, chemical tests and microbiological test.

The learning method used in the Deep Neural Network is supervised learning method. The input data 202 is Labeled and pairing of input data and output data is fed into the Deep Neural Network. As the data passes through input layer to Hidden layer 1, the nodes in the layer get trained on a distinct set of features. As the input data passes through Hidden layer 2, it aggregate and recombine features from Hidden layer 1 along with the features learned in that layer. Each node in each layer is associated with other nodes in non-linear manner. After passing through Hidden layers of the Deep Neural Network, the training dataset trains the Deep Neural Network to establish correlation between the pathological test result parameters and the chronic disease which is Liver Cirrhosis in the given example. It may be noted that the specific example of Liver Cirrhosis in FIG. 2 is given for illustrative purpose, while the method can be used to train the Deep Neural Network for any of the know chronic disease.

### Testing a New Patient Data to Predict a Probability of Occurrence of a Chronic Disease

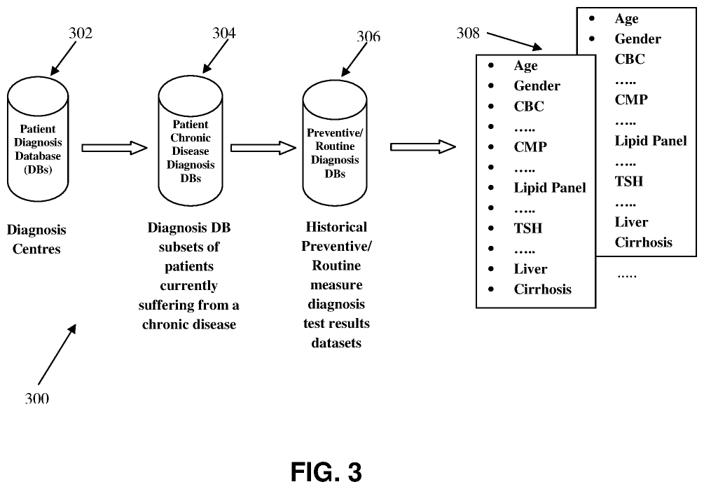

FIG. 3 is a process flow illustrating a system for using a person preventive or routine diagnostic report data for predicting a probability of occurrence of a chronic disease, in accordance with an embodiment of the present invention. A Patient Diagnosis Database 302 aggregates health records of patients from records of different diagnosis center. The health records of patients include historical/preventive pathological test result data of the patient. The patient diagnosis database (DBs) 302 at diagnosis center stores data from a number of diagnosed diseases corresponding to a number of different individuals or patients. For example, the data from the various health issues diagnosed can be stored in the patient diagnosis database 302. Long term outcomes are also captured and stored in the patient diagnosis database 302. According to an advantageous implementation, the data for each individual is anonymized prior to being stored in the common knowledge database 302 by removing any identifying information of the specific individual. This allows the anonymized data stored in the database 302 to be used for population studies and training the Deep Neural Network (DNN), without violating privacy concerns for the specific individuals.

The patient chronic disease diagnosis database 304 store the data related to diagnosis subsets of patients currently suffering from a chronic disease. In the database 304 all type of pathological test records of patients or individuals who are currently suffering from a chronic disease are stored. Such patient's historical routine pathological test results data can be learned by a Deep Neural Network and later can be used to predict for any new patient via just providing their similar pathological test results data.

The preventive/routine diagnosis database 306 store the routine pathological test results of the individuals or persons who are not suffering from a chronic disease but may suffer in future. The embodiment of the present invention collects those patients past routine/preventive laboratory test results 308 and feed into the Deep Neural Network that has been trained to detect correlation between changes in pathological test report parameters over a period of time and occurrence of a chronic disease. The Deep Neural Network analyses the historical preventive/routine diagnosis test result dataset of the user and analyzes the probability of occurrence of the chronic disease. The Deep Neural Network generates a prediction score for the user based on his past preventive pathological test result data. The prediction score signifies a probability that the user may have for the occurrence of the chronic disease.

In an embodiment, the routine preventive pathological test result include test result data on blood/serum analysis, urine analysis and stool analysis. The blood analysis result data may comprise test related to chemical pathology, hematology, anatomical pathology, medical microbiology, immunopathology, genetic pathology, genetic pathology, general pathology or clinical pathology. The blood analysis parameters may comprise but are not limited to CBC, CMP, LIPID Panel, TSH quantification, LFT, iron studies etc. The urine analysis test result data comprise but are not limited to ketoacids, electrolytes, acids, alkalines, proteins, glucose or amount of pathogen. The stool test pathological test result data may comprise but are not limited to microscopic examination, chemical tests and microbiological test.

In an embodiment of present invention, the trained DNN network either resides in a server or is distributed as a software installed on the client devices. The pathological test result data of the patient to be examined for the probability of having a chronic disease is entered through a user interface present on the client device. In an embodiment of present invention, the historical pathological test result data of the user can be entered using a user interface or it can be shared by the diagnostic centers. The user interface displays the end result of the analysis to the user in form of the prediction score.

In an embodiment of present invention, the trained DNN network either resides in a server or is distributed as a software installed on the client devices. In an embodiment of present invention, the historical pathological test result data of the user can be entered using a user interface or it can be shared by the diagnostic centers. The user interface displays the end result of the analysis to the user in form of the prediction score.

In an embodiment of present invention, the system and method notifies the physician, the healthcare provider and the patient about the probability of occurrence of the chronic disease. This will help the physician to formulate an effective treatment regime in good time for the treatment of high risk patient. In an embodiment, the system can notify the patient, the physician and the health care provider by sms or through a mobile application connected to the internet. This provide a support system for both patient and physician by improving the monitoring of a patient's health status and preventing further deterioration of the patient through chronic disease management. It also enables a patient to design individualized treatment action plan for the chronic disease.

The methods and processes described herein may have fewer or additional steps or states and the steps or states may be performed in a different order. Not all steps or states need to be reached. Some or all of the methods may alternatively be embodied in whole or in part in specialized computer hardware. The systems described herein may optionally include displays, user input devices (e.g., touchscreen, keyboard, mouse, voice recognition, etc.), network interfaces, etc.

The various illustrative logical blocks, modules, routines, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others.

I claim:

1. A system for predicting occurrence of a chronic disease in a patient, said system comprising:
a database for storing a dataset of historical pathological test result data of a plurality of patients diagnosed with a chronic disease;
a server having a Deep Neural Network in communication with the database, the DNN extracts the dataset from the database to identify a correlation between the changes in historical pathological data of a patient and the chronic disease;
a user interface to feed a new patient historical test result data into the trained Deep Neural Network;
wherein trained Deep Neural Network analyze change in historical test result data of the new patient data with the correlation to identify a prediction score for probability of occurrence of the chronic disease;
an application interface in the server to notify a user the prediction score.

2. The system of claim 1, wherein the dataset to train the Deep Neural Network are Labeled data.

3. The system of claim 1, wherein the training of Deep Neural Network is through supervised learning.

4. The system of claim 1, wherein the dataset is pre-processed before inputting into the Deep Neural Network.

5. The system of claim 4, wherein the pre-processed steps comprises Normalization and scaling of the dataset.

6. The system of claim 1, wherein the historical pathological data comprises test result data on blood/serum analysis, urine analysis or stool analysis.

7. The system of claim 6, wherein blood analysis result data may comprise test related to chemical pathology, hematology, anatomical pathology, medical microbiology, immunopathology, genetic pathology, genetic pathology, general pathology or clinical pathology.

8. The system of claim 1, wherein the application interface for notifying the user is a web-based application, web-browser, mobile application.

9. The system of claim 1, wherein the chronic disease may include but are not limited to prediction of neurological disorder, lungs disease, liver related disorder, cancer, gastrointestinal disorder, blood based disorder, heart related disorder.

10. The system of claim 1, wherein the user is a patient, a physician or a healthcare provider.

11. The system of claim 1, wherein the changes in historical pathological data includes changes in one or more analytes or parameters in blood/serum of the patient.

12. A method for predicting occurrence of a chronic disease in a patient using historical pathological test result data, the method comprising:
training a Deep Neural Network with a dataset comprising historical pathological test result data of a plurality of patients that have been diagnosed with a chronic disease,
said Deep Neural Network analyze the data and learns to identify a correlation between the changes in historical pathological data of a patient and the chronic disease;
feeding a new patient historical test result data into the trained Deep Neural Network wherein trained Deep Neural Network analyze change in historical test result data of the new patient data with the correlation to generate a prediction score for identifying a probability of occurrence of the chronic disease;
notifying a user the prediction score for probability of occurrence of the chronic disease.

13. The method of claim 12, wherein the dataset to train the Deep Neural Network are Labeled data.

14. The method of claim 12, wherein the user is notified by sms, email or through a web-based application.

15. The method of claim 12, wherein the training of Deep Neural Network is through supervised learning and inputs to the Deep Neural Network of Labeled data.

16. The method of claim 12, wherein the dataset is pre-processed before inputting into the Deep Neural Network.

17. The method of claim 16, wherein the pre-processed steps comprises Normalization and scaling of the dataset.

18. The method of claim 12, wherein the changes in historical pathological data includes changes in one or more analytes or parameters in blood/serum, urine or stool sample of the patient.

19. The method of claim 12, wherein blood analysis result data may comprise test related to chemical pathology, hematology, anatomical pathology, medical microbiology, immunopathology, genetic pathology, genetic pathology, general pathology or clinical pathology.
